(19) United States
(12) Patent Application Publication
(10) Pub. No.: US 2015/0246865 A1
(43) Pub. Date: Sep. 3, 2015

(54) PROCESS FOR MAKING LEVULINIC ACID

(71) Applicant: ARCHER DANIELS MIDLAND COMPANY, Decatur, IL (US)

(72) Inventors: Alexandra Sanborn, Lincoln, IL (US); Thomas Binder, Decatur, IL (US)

(73) Assignee: ARCHER DANIELS MIDLAND COMPANY

(21) Appl. No.: 14/705,116

(22) Filed: May 6, 2015

Related U.S. Application Data
(63) Continuation of application No. 14/358,373, filed on May 15, 2014.
(60) Provisional application No. 61/584,890, filed on Jan. 10, 2012.

(51) Int. Cl. C07C 51/31

(52) U.S. Cl. CPC C07C 51/31

(57) ABSTRACT

A process is described wherein a feed of a six-carbon carbohydrate-containing material or of a furanic dehydration product from a six-carbon carbohydrate-containing material or of a combination of these is supplied to a reactor in a controlled manner over time, and the feed is acid-hydrolyzed to produce levulinic acid. In certain embodiments, derivatives of the levulinic acid are prepared.

### CROSS REFERENCE TO RELATED APPLICATIONS

This application is a continuation of U.S. Ser. No. 14/358,373, which was filed on May 15, 2014 from Patent Cooperation Treaty Application No. PCT/US12/66710 filed Nov. 28, 2012, which in turn claimed the benefit of U.S. Ser. No. 61/584,890, filed Jan. 10, 2012.

### BACKGROUND OF THE INVENTION

The present invention is concerned with processes for making levulinic acid and derivatives thereof from sugars, and particularly but without limitation, from sugars from biomass.

The bulk of energy needs and the vast majority of synthetic products and chemicals have historically been sourced from fossil fuels. As fossil fuels have become more scarce or less accessible, and as the financial, environmental and other societal costs associated with locating, recovering and using fossil fuels have increased in recent years, however, significant research efforts have been undertaken to meet energy needs and produce chemical products from biomass that could replace the fossil fuel-based materials.

Biomass is the only renewable source of fixed carbon, which is essential for the production of liquid hydrocarbons and chemicals. Over 150 billion tons of biomass are produced per year through photosynthesis, yet only 3-4% is used by humans for food and non-food purposes. Low value agricultural and forestry residues, grasses and energy crops are preferred sources of biomass for making biobased or bioderived fuels and chemical products, and provide an opportunity to make the transportation fuels and chemical products that are needed from renewable resources.

The National Renewable Energy Laboratory (Denver, USA) has identified levulinic acid as one of a number of key sugar-derived platform chemicals that can be produced from biomass. Levulinic acid can be used to produce a variety of materials for a variety of uses, including succinic acid, 1,4-butanediol, 1,4-pentanediol, tetrahydrofuran, gamma valerolactone, ethyl levulinate and 2-methyl-tetrahydrofuran, for example, for producing resins, polymers, herbicides, pharmaceuticals and flavoring agents, solvents, plasticizers, antifreeze agents and biofuels/oxygenated fuel additives.

Rackemann and Doherty, “The Conversion of Lignocellulosics to Levulinic Acid”, Biofuels, Bioproducts & Biorefining, 5:198-214 (2011) provides an overview of current and potential technologies which had been publicly identified or suggested, for producing levulinic acid from lignocellulosics. The “most promising” commercial process according to the reviewers utilized the Biofine™ technology developed by Fitzpatrick (and described for example in U.S. Pat. No. 5,608,105), involving a two-stage acid-catalyzed process wherein in a first, plug flow reactor a carbohydrate-containing material (primary sludges from paper manufacture, waste paper, waste wood, agricultural residues such as corn husks, corn cobs, rice hulls, straw, bagasse, food processing wastes from corn, wheat oats and barley) is dehydrated to 2,5-hydroxymethylfurfural (HMF) at from 210 to 230 degrees Celsius for less than 30 seconds, and then levulinic acid is produced in a second reactor at 195 to 215 degrees Celsius for 15 to 30 minutes. The reviewers conclude that further improvements must be made, however, for the cost-effective production of levulinic acid from biomass:
“The key to improving the yield and efficiency of levulinic acid production from biomass lies in the ability to optimize and isolate the intermediate products at each step of [a multi-step] reaction pathway and reduce re-polymerization and side reactions. New technologies (including the use of microwave irradiation and ionic liquids) and the development of highly selective catalysts would provide the necessary step change for the optimization of key reactions. A processing environment that allows the use of biphasic systems and/or continuous extraction of products would increase reaction rates, yields and product quality.”

Consequently, on a consideration of the body of published work related to the production of levulinic acid from biomass sources, the direction for further development provided by these reviewers was toward more complex, multistep processes to address the “major challenge” posed by “the complex nature of the biomass substrate”, “the presence of non-cellulose components” and the fact that the conversion from biomass to levulinic acid proceeds “through a number of pathways involving multiple steps and intermediates”, Rackemann and Doherty at page 210.

### SUMMARY OF THE INVENTION

The present invention relates in one aspect to an improved process for making levulinic acid, wherein a feed comprised of a six-carbon carbohydrate-containing material or a furanic material derived therefrom from a six-carbon carbohydrate-containing material or a combination of these is supplied to a reactor in a controlled manner over time and acid hydrolyzed in the reactor to produce a product including levulinic acid. In an alternate embodiment, the product further includes a derivative of levulinic acid.

We have found that by providing a quantity of six-carbon sugars (whether from biomass or from another source) and/or corresponding furanic dehydration products from those sugars (which includes the ether and ester derivatives of the immediate dehydration product hydroxymethylfurfural (or HMF), as further described below) to a levulinic acid manufacturing process in a controlled manner over time—whether in increments, semi-continuously or continuously at an overall or continuously controlled rate of addition, or indeed through any mode of addition wherein the six-carbon sugars and/or their corresponding furanic dehydration products are input over time up to a desired feed level—then levulinic acid and/or its derivatives can be produced in relatively higher yields from that amount of feed as compared to the circumstance wherein the same amount of the feed is added all at once. Further, where six-carbon sugars are fed to the reactor, the levulinic acid can be efficiently produced without the necessity of recovering a furanic dehydration product intermediate (hydroxymethylfurfural, for example) for separate processing—and in fact, the levulinic acid can be produced with preferably low levels of unconverted furanic dehydration products, without requiring the development and/or use of “highly selective catalyst(s)” tailored to the conversion of sugars to furanic dehydration intermediate products, or of the furanic dehydration products to levulinic acid or for both conversions.

Parenthetically, by “furanic dehydration product”, it is not intended that this terminology excludes the same materials as made by means other than dehydration of six-carbon sugars. For example, HMF may be prepared enzymatically from these sugars, and it is intended that “furanic dehydration product” would encompass HMF made in this fashion.

### BRIEF DESCRIPTION OF THE DRAWING

FIG. 1 is a graph of the percentage molar yields of levulinic acid achieved experimentally with dextrose (glucose) using the controlled substrate addition method of the present invention, as a function of the percentage of dissolved solids cumulatively fed to the reactor.

### DETAILED DESCRIPTION OF CERTAIN EMBODIMENTS

Many common materials consist partially or fully of carbohydrates. The most abundant hexose or C6 sugar found in nature is glucose, available in the polysaccharide form as starch or cellulose (in biomass) and in the disaccharide form as sucrose (derived from glucose and fructose). Other naturally occurring hexoses include galactose and mannose present in the hemicellulose component of biomass, and fructose which along with glucose is found in many foods and is an important dietary monosaccharide.

Lignocellulosics are a particular type of biomass from which C6 sugars can be obtained, being comprised of cellulose, hemicellulose and lignin fractions. Cellulose is generally the largest fraction in biomass, and derives from the structural tissue of plants, consisting of long chains of beta glucosidic residues linked through the 1,4 positions. These linkages cause the cellulose to have a high crystallinity and thus a low accessibility to the enzymes or acid catalysts which have been suggested for hydrolyzing the cellulose to C6 sugars or hexoses. Hemicellulose by contrast is an amorphous heteropolymer which is easily hydrolyzed, while lignin, an aromatic three-dimensional polymer, is interspersed among the cellulose and hemicellulose within a plant fiber cell and lends itself to still other process options.

Parenthetically in regards to the lignin fraction, the materials understood as encompassed within the term “lignin” and the method by which lignin content has been correspondingly quantified in a biomass have historically depended on the context in which the lignin content has been considered, “lignin” lacking a definite molecular structure and thus being determined empirically from biomass to biomass. In animal science and agronomy, in considering the digestible energy content of lignocellulosic biomasses, for example, the amount of lignin in a given biomass has more commonly been determined using an acid detergent lignin method (Goering and Van Soest, Forage Fiber Analyses (Apparatus, Reagents, Procedures, and Some Applications), Agriculture Handbook No. 379, Agricultural Research Service, United States Dept of Agriculture (1970); Van Soest et al., “Methods for Dietary Fiber, Neutral Detergent Fiber, and Nonstarch Polysaccharides in Relation to Animal Nutrition”, J. Dairy Sci., vol. 74, pp 3583-3597 (1991)). In the paper and pulp industry, by contrast, the amount of lignin in a given biomass has been conventionally determined by the Klason lignin method (Kirk and Obst, “Lignin Determination”, Methods in Enzymology, vol 16, pp.: 89-101 (1988)). For purposes of the present invention, where a lignocellulosic biomass is contemplated for providing the C6 sugars feedstock, the lignocellulosic biomasses of most interest will be those having at least a lignin content consistent with mature temperate grasses having relatively low nutritive value for ruminants and which consequently are diverted to other uses in the main, such grasses typically being characterized by 6% or more of acid detergent insoluble materials (on a dry weight basis).

As already observed above, the hemicellulose fraction of biomass can be a source of C6 sugars for the inventive process. Those skilled in the art will appreciate, however, that where lignocellulosic biomasses are used for providing at least some portion of the C6 sugars fed to the inventive process, the hemicellulose fraction in being comprised mostly of xylan (though containing also arabinan, galactan and mannan) can be a substantial source of C5 sugars (or pentoses), as well. While forming no part of the present invention, these C5 sugars can also be converted to the same desired levulinic acid and levulinic acid derivative products thereof through a variety of known processes.

In particular, and as further described in U.S. Pat. No. 7,265,239 to Van De Graaf et al. as well as the previously-cited Rackemann and Doherty review at page 203, furfural can be obtained as the acid-catalyzed dehydration product from the pentoses in a hemicellulose fraction of biomass, the furfural can be catalytically reduced by the addition of hydrogen to furfuryl alcohol, and furfuryl alcohol can be converted to levulinic acid and alkyl levulinates. In the '239 Van De Graaf patent, furfuryl alcohol and water are converted to levulinic acid with the use of a porous strong acid ion-exchange resin, or furfuryl alcohol with an alkyl alcohol are converted to an alkyl levulinate. Still earlier references describe other means for converting the pentoses in the hemicellulosic fraction of biomass into levulinic acid and/or its derivatives, by means of furfural and furfuryl alcohol, see, for example, U.S. Pat. Nos. 2,738,367; 4,236,012; 5,175,358; 2,763,665; 3,203,964; and 3,752,849.

One of the challenges enumerated by the Rackemann and Doherty article for making a bioderived levulinic acid product on a commercial basis concerns the complex nature of biomass starting materials in relation to the presence of cellulosic, hemicellulosic and lignin fractions therein, but presumably also in relation to the variety of biomasses that exist and the variability of a given biomass based on harvesting or collection methods and circumstances, storage conditions and the like. Certainly this makes a great deal of sense, in considering the extent to which small compositional differences in a feed can affect the performance of the “highly selective catalysts” contemplated by the article for a future commercial bioderived levulinic acid process.

A benefit of the process of the present invention is that, as demonstrated by the examples which follow, a variety of six-carbon carbohydrate-containing materials can be readily accommodated, along with hydroxymethylfurfural from the acid-catalyzed dehydration of C6 sugars and the more stable derivatives of HMF that have been proposed for use as an alternative feedstock for chemical synthesis, see, e.g., U.S. Pat. No. 7,317,116 and US 2009/0156841 to Sanborn et al. (HMF ethers and HMF esters), both references now being incorporated by reference herein.

In one embodiment, a lignocellulosic biomass is used to provide the six-carbon carbohydrate-containing material. More particularly, a cellulosic fraction of the biomass can be hydrolyzed to provide some combination of hexose monomers and oligomers, HMF and HMF derivatives, according to any of the various known processes for fractionating a biomass and hydrolyzing the cellulosics to hexoses and hexose-derivative products. One such process, of course, is the Biofine process described in U.S. Pat. No. 5,608,105 to Fitzpatrick. In another embodiment, both of the cellulosic and hemicellulosic fractions are used, with the pentoses from the hemicellulosic fraction being converted as described above to furfural and then to furfuryl alcohol, before being fed into the instant levulinic acid process either alone or in combination with the hexoses and hexose-derivative products (such as HMF, HMF esters, HMF ethers) from the cellulosic fraction.

Because of the differences in the cellulosic, hemicellulosic and lignin fractions of biomass, as well as considering other lesser fractions present in various biomasses to different degrees, as related in U.S. Pat. No. 5,562,777 to Farone et al., “Method of Producing Sugars Using Strong Acid Hydrolysis of Cellulosic and Hemicellulosic Materials”, a number of processes have been developed or proposed over the years to fractionate lignocellulosic biomasses and hydrolyze the cellulosic and hemicellulosic fractions to provide usable hexose and pentose synthesis feeds. A commonly-assigned Patent Cooperation Treaty application published as WO 2011/097065, incorporated by reference herein, describes another method by which a lignocellulosic biomass can be fractionated and the cellulosic and hemicellulosic fractions hydrolyzed to provide the C6 and optional C5 sugars, respectively, that can be used in the present levulinic acid process.

In still another embodiment, glucose, fructose or a combination thereof comprise the six-carbon carbohydrate containing feed to the process.

In particular, responsive to changes in the demand for high fructose corn syrup (HFCS), one or more of the commonly used HFCS 42 (about 42% fructose and 53% glucose of the total sugars in a water-based syrup; used in many food products and baked goods), HFCS 55 (about 55% fructose and 42% glucose, used mainly in soft drinks) and HFCS 90 (about 90% fructose and 10% glucose, used primarily as a blendstock with HFCS 42 to make HFCS 55) can be diverted to make levulinic acid and other valuable derivative products, and thus provide improved asset utilization of HFCS production facilities and/or an opportunity for improved margins for a producer of HFCS.

In still another embodiment, the six-carbon sugars can be or include unconverted sugars recovered from another process which utilizes hexose sugars as a feed, for example, any of the numerous processes which have been proposed for making hydroxymethylfurfural and/or derivatives thereof from such sugars. In particular, where it may be desired to produce both levulinic acid (and/or products made from or based upon levulinic acid) and HMF (and/or other products made from or based upon HMF), the residual sugars product can be used as recovered from the HMF manufacturing process described in the commonly-assigned U.S. Provisional patent Application filed concurrently herewith, entitled “Process For Making Hydroxymethylfurfural With Recovery Of Unreacted Sugars Suitable For Direct Fermentation To Ethanol”, such application being incorporated by reference herein.

As mentioned previously, levulinic acid (and its derivatives, such as the levulinate esters for example) has been contemplated for use in making a number of different products for a variety of different uses, for example, succinic acid, 1,4-butanediol, 1,4-pentanediol, tetrahydrofuran, gamma valerolactone, ethyl levulinate and 2-methyl-tetrahydrofuran for producing resins, polymers, herbicides, pharmaceuticals and flavoring agents, solvents, plasticizers, antifreeze agents and biofuels/oxygenated fuel additives. A detailed description of the methods which have been suggested for making these various valuable derivatives need not be undertaken herein, but an example of a further method for using the levulinic acid would be to spray oxidize the same to form succinic acid, according to Patent Cooperation Treaty Application Serial No. PCT/US12/52641, filed Aug. 31, 2011 for “Process for Producing Both Biobased Succinic Acid and 2,5-Furandicarboxylic Acid”. In this particular application, sugar dehydration products inclusive of levulinic acid and HMF—or derivatives of the same, such as the levulinate esters and HMF esters, that will oxidize to the same succinic acid and FDCA products—can be concurrently spray oxidized to provide both biobased succinic acid and FDCA in the presence of a Mid-Century type Co/Mn/Br catalyst under oxidation conditions. Consequently, in the context of the present invention, should some HMF or HMF esters remain in the levulinic acid product, that product can nevertheless be directly processed as a feed in the indicated spray oxidation process to provide valuable derivative products therefrom.

A process for making levulinic acid according to the present invention comprises, in one embodiment, supplying a feed including a six-carbon carbohydrate-containing material or a furanic dehydration product from a six-carbon carbohydrate-containing material or a combination of these to a reactor in a controlled manner over time up to a desired feed level, and then acid hydrolyzing the feed in the reactor to produce a product including levulinic acid. In an alternate embodiment, the product further includes a derivative of levulinic acid.

We have found that by providing the hexoses and/or the HMF, HMF esters and ethers in a controlled manner over time—whether in increments, semi-continuously or continuously at an overall or continuously controlled rate of addition, or indeed through any mode of addition wherein the six-carbon sugars and/or their corresponding furanic dehydration products are input over time up to a desired feed level—then levulinic acid and/or its derivatives can be produced in relatively higher yields or proportions, as compared to the circumstance wherein these materials are added all at once for a batchwise process or continuously at the ending feed level for a continuous process. Further, where six-carbon sugars are fed to the reactor, the levulinic acid can be efficiently produced without the necessity of recovering a furanic dehydration intermediate (hydroxymethylfurfural, for example) for separate processing—and in fact, the levulinic acid can be produced with preferably low levels of unconverted furanic dehydration products, without requiring the development and/or use of “highly selective catalyst(s)” tailored to the conversion of sugars to furanic dehydration intermediate products, or of the furanic dehydration products to levulinic acid or for both conversions.

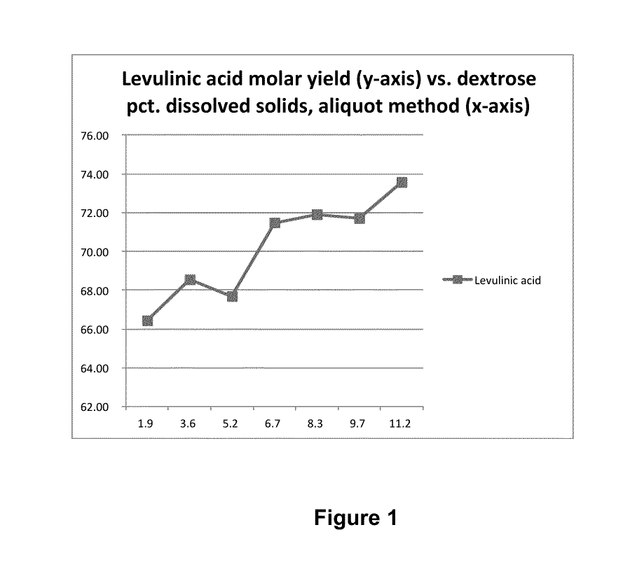

The difference in the molar yield of levulinic acid which can be achieved for a given quantity of feed can vary based on the nature of the feed, reaction conditions, feed concentration and the amount of time over which feed is supplied to the reactor (as shown clearly by the examples which follow), but in general a yield improvement on a molar basis of 5 percent or more, especially 10 percent or more and even 20 percent and greater is achievable by introducing the feed over a period of time rather than at once. Moreover, as can be seen from several examples, by introducing and hydrolyzing the feed incrementally or over time generally, a greater throughput of the feed should be possible, further increasing the productivity of the process. Preferably, at least five percent more by weight of hexoses, HMF and HMF ester and ether derivatives can be reacted in a given batch or over a given run time in a continuous process, and more preferably still at least ten percent more by weight can be processed, as compared to the circumstance wherein the same quantity of feed is introduced at once. As may be seen from FIG. 1, with the controlled addition method described herein increased concentrations of dextrose were observed experimentally to coincide with higher overall molar yields to the levulinic acid product.

Through controlling the addition of feed to the reactor, low levels of unconverted residual HMF and HMF derivatives can be achieved if desired, as the hexoses, HMF and HMF derivatives are quickly completely converted on introduction to levulinic acid and/or its derivatives within the larger acidic matrix. Preferably, where low levels of unconverted HMF and HMF derivatives are sought, the resultant levulinic acid product contains not more than 3 percent by weight of furanic materials in relation to the amount of levulinic acid and levulinic acid derivatives formed, more preferably containing not more than 2 percent and most preferably not more than 1.5 percent of the total levulinic acid and derivatives formed. Alternatively, of course, where the levulinic acid product is to be supplied as a feed for concurrently producing both FDCA and succinic acid according to the process of the Patent Cooperation Treaty Application Serial No. PCT/US12/52641, higher furanic contents can be obtained through introducing the feed material over a shorter timeframe given the same hydrolysis conditions otherwise, or through the use of reduced amounts of sulfuric acid.

The reaction can be conducted in an otherwise conventional manner, in a batchwise, semi-batch or continuous mode, using such homogeneous or heterogeneous acid catalysts and under reaction conditions such as have been described or found useful previously for converting hexoses, HMF and HMF ester and ether derivatives to levulinic acid and its derivatives. Preferred and optimized conditions of catalyst, catalyst loading, temperature, feed rate or increment sizing, feed cycle time (for continuous feed (whether constant, variable or ramped)) or feed increment interval (for feeding in increments) can be expected to vary dependent on the particular feed chosen. In general, feed rates and resultant overall feed cycle times can, for the same quantity of a given feed and under the same other conditions, provide some variation in product distribution and yields, and the overall process can be optimized around a feed rate (or a range of feed rates) and an overall feed cycle time (or range of times) based on the costs and benefits of longer overall cycle times versus shorter.

In one embodiment, however, HFCS 90 can be converted to levulinic acid in the presence of from 0.1 to 0.5 grams of sulfuric acid per gram of sugar substrate and at a temperature of from 150 degrees Celsius and especially from 160 degrees Celsius, up to 210 degrees Celsius but especially 185 degrees Celsius or less. A feed rate of HFCS 90 in such an embodiment can be 2.5 percent of the feed per minute, by weight. In such an embodiment, the sulfuric acid is preferably supplied to the reactor and slowly preheated to the desired reaction temperature before fructose syrup begins to be supplied to the reactor.

In another embodiment, water and concentrated sulfuric acid can be supplied in order to provide a beginning sulfuric acid concentration of from 3 to 3.5 weight percent in a 1 L reactor, and the contents of the reactor can be brought to a temperature of 180 degrees Celsius. A fructose solution containing from 30 percent to 50 percent fructose in water is pulsed into the reactor in one minute increments at 7 mL/minute, with successive increments of the feed being pulsed in, in from 5 to 9 minute intervals, until the feed is completely input to the reactor over a total of from 4 to 6 hours. As the first feed increment enters the reactor, the reactor is characterized as having an effective sugar concentration of from 0.6 to 1 percent by weight of the total reaction mass. As the last feed increment enters the reactor, the effective sugar concentration in the reactor is from 0.2 to 0.5 percent by weight of the reactor contents. The corresponding concentration of sulfuric acid in the reactor contents as the last feed increment is added is from 0.7 to 1.5 percent by weight.

The present invention is more particularly illustrated by the examples below:

### EXAMPLES

### Example 1

A solution of deionized water (40.22 grams), hydroxymethylfurfural (98% HMF by distillation, 0.73 grams) and 630 μL of sulfuric acid (0.3M initial concentration) was heated in a 75 mL Parr reactor vessel to 180 degrees Celsius over a period of 25 minutes. The solution was maintained at this temperature for five minutes with continuous stirring at 850 rpm, and then was cooled rapidly by immersion in an ice bath for from 3-4 minutes. A sample was collected of the reactor contents for HPLC analysis, and a further increment of about 0.7 grams of HMF was added to the reactor, with heating again to 180 degrees Celsius, holding at 180 degrees for five minutes, rapid cooling and withdrawal of a sample for analysis. Two additional HMF increments were added and reacted in the same fashion, until the total HMF added to the reactor on a dry solids basis was about 6.85 percent by weight. Analysis of the samples associated with each increment of HMF feed showed that the overall yield of levulinic acid increased with each successive, fully reacted HMF increment, from about 74 mol percent to about 81 mol percent to about 82 mol percent and finally to about 85 mol percent.

The HPLC apparatus used consisted of an LC-20AT pump (Shimadzu, Tokyo, Japan), a CTO-20A column oven (Shimadzu, Tokyo, Japan), an RID detector (Shimadzu, Tokyo, Japan) and an SPD-10A ultraviolet detector (Shimadzu, Tokyo, Japan). The chromatographic data was acquired using the CBM-20A system controller (Shimadzu, Tokyo, Japan). The separations of sugars, formic and levulinic acids were performed on a Shodex Sugar column (8.0 mmID×300 mmL). The separations of 5-hydroxymethyl furfural and 2-furaldehyde were performed on a Waters Symmetry C18 column (150 mm×4.6 mm).

The mobile phase chosen for the sugar column was 5 mM Sulfuric Acid. The flow-rate of the mobile phase was 0.8 mL/min. All experiments were carried out at 50.0° C. RID was used for detection. The mobile phase chosen for the Waters Symmetry C18 Column was a gradient with acetonitrile and water. All experiments were carried out at 40.0° C.

The quantitative analyses were performed by using external standards based on area of peak. The method was calibrated using a series of 5 external standards of known concentrations.

Samples were diluted. Samples for the sugar analysis were diluted 1:1 using the mobile phase and filtered with a 0.2 μm PVFD filter. Samples for the furan analysis were diluted using 10% acetonitrile and filtered with a 0.2 μm PTFE filter. Dilutions depended on the theoretical amount of furans.

### Comparative Example 1

For comparison to the results obtained in Example 1, about 6.4 percent of HMF on a dry solids basis was combined with the water and sulfuric acid at one time, in a single addition. The solution was heated to 180 degrees Celsius over 25 minutes as in Example 1, then held at 180 degrees for five minutes and rapidly cooled. A sample of the reactor contents was taken and analyzed as described in Example 1, and showed levulinic acid was produced at about 75 mol percent. Some formation of black solids (humins) was also noted.

### Example 2

A concentrated solution of HFCS 90 was combined in a first increment with 0.3 M sulfuric acid solution, to provide about 1.5 percent of fructose in the acid solution on a dry solids basis. The solution was heated to 180 degrees Celsius gradually, over a period of about 25 minutes. This temperature was held for 2.5 minutes, followed by rapid cooling of the reactor vessel in an ice bath for from one to two minutes. A sample was withdrawn for analysis, and further increments were added, heated, held at temperature and cooled for sampling at dry solids loadings of about 2.9 percent (2^{nd} increment), 4.3 percent (3^{rd}), 5.6 percent (4^{th}), 6.9 percent (5^{th}), 8.1 percent (6^{th}) and 9.2 percent (7^{th}). Analysis of the reactor contents showed a molar yield of levulinic acid in the reactor contents increasing from the first increment to a dry solids loading of 5.6 percent, from less than 70 percent to about 80 percent. The overall yield of levulinic acid on a molar basis thereafter declined slightly, to about 73 percent after 9.2 percent of sugars had been processed on a dry solids basis; concurrently, the yield of the HMF intermediate increased from about 1.0 mol % to about 4.0-4.1 mol %. Furfural levels were from 1.0 to 0.5 mol %, and residual glucose/levoglucosan levels declined from about 3.4 mol % to less than 0.5 mol %.

### Example 3

A concentrated solution of HFCS 90 was combined in a first increment with 0.3 M sulfuric acid solution. The solution was heated to 180 degrees Celsius gradually, over a period of about 25 minutes. This temperature was held for 6 minutes, followed by rapid cooling of the reactor vessel in an ice bath for from one to two minutes. A sample was withdrawn for analysis, and five further increments of 0.9 grams each (on a dry solids basis) were added, heated, held at temperature and cooled for sampling up to a combined total dry solids loading of about 7 percent. The molar yield for levulinic acid after incrementally adding the 7 percent sugars on a dry solids basis was 74 percent.

### Comparative Example 3

The same amount of HFCS 90 was added to 0.3 M sulfuric acid as in Example 3, but in a single addition. After heating to 180 degrees Celsius and holding for 6 minutes at this temperature, the reaction mixture was rapidly cooled in an ice bath. Analysis of the reactor contents showed levulinic acid was produced at a 55 percent molar yield, almost 20 percentage points lower than with the incremental addition mode.

### Example 4

A concentrated solution of HFCS 90 was combined in a first increment with 0.3 M sulfuric acid solution. The solution was heated to 180 degrees Celsius gradually, over a period of about 25 minutes. This temperature was held for 6 minutes, followed by rapid cooling of the reactor vessel in an ice bath for from one to two minutes. A sample was withdrawn for analysis, and four further increments of 0.9 grams each (dry solids basis) were added, heated, held at temperature and cooled for sampling up to a combined total dry solids loading of about 5 percent. The molar yield for levulinic acid after incrementally adding the 5 percent sugars on a dry solids basis was 87 percent.

### Comparative Example 4

The same amount of HFCS 90 was added to 0.3 M sulfuric acid as in Example 4, but in a single addition. After heating to 180 degrees Celsius and holding for 6 minutes at this temperature, the reaction mixture was rapidly cooled in an ice bath. Analysis of the reactor contents showed levulinic acid was produced at a 66 percent molar yield, again almost 20 percentage points lower than with the incremental addition mode.

### Examples 5-9

A series of experiments were performed with continuous gradual addition of the same amount of dextrose over different overall feed cycle times, but otherwise identical conditions. For these examples, a total of 9 percent of dextrose on a dry solids basis was added over a period of time to a sulfuric acid solution providing 0.65 grams of sulfuric acid per gram of the total dextrose feed, and containing 0.17 grams of AlCl₃ per gram of the total dextrose feed to promote the isomerization of the dextrose to the more-readily converted fructose. The combination was gradually heated to 180 degrees Celsius, held at that temperature for 10 minutes, then rapidly cooled, sampled and analyzed. Feed cycle times ranged from 1 minute, to 2 minutes, to 7 minutes, 20 minutes and 40 minutes. Levulinic acid yield on a mol percent basis was 46 percent for the one minute feed cycle time, 51 percent for the two minute feed cycle time, 59 percent for a seven minute continuous addition feed cycle, 62 percent for a twenty minute cycle and 63 percent for a forty minute cycle.

### Examples 10-13

The same overall approach was taken for 9 percent of fructose on a dry solids basis as was used for Examples 5-9, except that sulfuric acid content was adjusted to 0.54 grams of sulfuric acid per gram of fructose and no AlCl3 was used. Overall feed cycle times were 1.25 minutes, 5 minutes, 20 minutes and 40 minutes. Corresponding levulinic acid yields on a mol percent basis were 47, 52, 51 and 65 percent, respectively.

### Example 14

A solution of 40 grams of water, 1800 μL of sulfuric acid (providing 0.66 grams of acid per gram of dextrose) and 0.8 grams of AlCl₃ (providing 0.16 grams per gram of dextrose) was heated to 180 degrees Celsius with stirring at 850 rpm. A 25% aqueous solution of dextrose was pumped into the reactor at 1.0 mL/minute for 20 minutes, to provide a total of about 8.1 percent of dextrose on a dry solids basis. Samples were pulled during the addition process at 10, 15 and 20 minutes and these were analyzed. Levulinic acid molar yield in the reaction mixture after 10 minutes of addition was 62 percent, while being 64 percent after 15 and 20 minutes of substrate addition.

### Example 15

A solution of deionized water (40.3 g), HFCS 90 (0.94 g) and 630 μL of sulfuric acid was heated in a 75 mL Parr reactor vessel to 180 degrees Celsius over 25 minutes. The solution was maintained at this temperature for 6 minutes with continuous stirring at 850 rpm, and then cooled rapidly by immersion into an ice bath. A sample was withdrawn for analysis, and the cycle was repeated for seven additional increments of HFCS 90 until the total amount of sugars added to the reactor on a dry solids basis was about 11.4 percent. The molar yields of various components in the reaction mixture were as shown in Table 1, in percents:

TABLE 1

| | | | Levulinic | | Glucose and |
|---|---|---|---|---|---|
| Increment | HMF | Furfural | Acid | Fructose | Levoglucosan |
| 1 | 1 | 1 | 74 | 0 | 2 |
| 2 | 1 | 1 | 79 | 0 | 1 |
| 3 | 1 | 1 | 89 | 0 | 0 |
| 4 | 1 | 1 | 87 | 0 | 0 |
| 5 | 2 | 1 | 74 | 0 | 0 |
| 6 | 4 | 1 | 84 | 0 | 0 |
| 7 | 8 | 0 | 71 | 0 | 0 |
| 8 | 9 | 0 | 68 | 0 | 0 |

### Example 16

A 1 liter autoclave reactor was charged with 300 grams of 3.8 weight percent sulfuric acid solution (in water). The reactor system was assembled and heated to 180 degrees Celsius. After the set temperature was reached, 300 grams of 33 weight percent fructose solution in water was pulsed into the reactor over time, by feeding the fructose solution for 1 minute intervals and then holding at the 180 degree Celsius temperature for five minutes before adding in the next 1 minute increment of fructose solution. After all of the fructose solution was added, the reactor contents were held at 180 degrees Celsius for another thirty minutes, after which the reactor was cooled to room temperature and the contents filtered. About 15 grams of char were removed from the filtrate, and the remainder was analyzed. The sample (596 grams) contained 5.16 weight percent of levulinic acid, 2.23 weight percent of formic acid, 0.02 weight percent of HMF, 0.01 weight percent of furfural, and sugars were not detected. The molar percentage yield of levulinic acid was 78 percent.

What is claimed is:

1. An improved process for making levulinic acid in higher yields from acid hydrolyzing a given quantity of a six-carbon carbohydrate-containing material or of a furanic dehydration product from a six-carbon carbohydrate-containing material or of a combination of these, wherein the improvement comprises supplying the given quantity to the process to be acid hydrolyzed in a controlled manner over time.

2. A process according to claim 1, further comprising forming a levulinic acid derivative from the levulinic acid.

3. A process according to claim 2, wherein the process produces a product containing not more than 3 percent by weight of combined unconverted furanic dehydration products and derivatives in relation to the amount of levulinic acid and levulinic acid derivatives found therein.

4. A process according to claim 3, wherein the product contains not more than 2 percent by weight of combined unconverted furanics.

5. A process according to claim 4, wherein the product contains not more than 1.5 percent by weight of combined unconverted furanics.

6. A process according to claim 1, wherein the process produces a product containing not more than 3 percent by weight of combined unconverted furanic dehydration products and derivatives in relation to the amount of levulinic acid and levulinic acid derivatives found therein.

7. A process according to claim 6, wherein the product contains not more than 2 percent by weight of combined unconverted furanics.

8. A process according to claim 7, wherein the product contains not more than 1.5 percent by weight of combined unconverted furanics.

9. A process according to claim 1, wherein the given quantity is supplied to the reactor in increments spaced over time.

10. A process according to claim 1, wherein the given quantity is supplied to the reactor in a plurality of intervals of continuous feeding spaced over time.

11. A process according to claim 1, wherein the given quantity is supplied to the reactor continuously over time.

12. A process according to claim 9, wherein the given quantity is supplied over a period of time ranging from 40 minutes to 6 hours.

13. A process according to claim 10, wherein the given quantity is supplied over a period of time ranging from 40 minutes to 6 hours.

14. A process according to claim 11, wherein the given quantity is supplied over a period of time ranging from 40 minutes to 6 hours.

15. A process according to claim 1, wherein the reactor is supplied with fructose, glucose or both of fructose and glucose.

16. A process according to claim 15, wherein the reactor is supplied with high fructose corn syrup.

17. A process according to claim 1, wherein the reactor is supplied with a cellulosic fraction of a lignocellulosic biomass.

18. A process according to claim 17, wherein the feed further comprises furfuryl alcohol.

19. A process according to claim 18, further comprising the steps of: fractionating a lignocellulosic biomass into lignin-containing, cellulosic and hemicellulosic fractions; dehydrating pentoses in the hemicellulosic fraction to provide furfural; and converting furfural to furfuryl alcohol.
